(19) United States
(12) Patent Application Publication
(10) Pub. No.: US 2004/0193378 A1
(43) Pub. Date: Sep. 30, 2004

(54) Method and system for registration, identifying and processing of drug specific data

(75) Inventors: J. Gut, Grellingen (CH); Dario Bagatto, Allschwil (CH)

Correspondence Address: SIDLEY AUSTIN BROWN & WOOD LLP, 787 Seventh Avenue, New York, NY, 10019, US

(73) Assignee: TheraStrat AG

(21) Appl. No.: 10/806,306

(22) Filed: Mar. 22, 2004

Related U.S. Application Data
(63) Continuation-in-part of application No. 10/005,666, filed on Nov. 2, 2001.

(30) Foreign Application Priority Data
Nov. 3, 2000 (CH) 00 811 026.4

(51) Int. Cl. G06F 19/00; G01N 33/48; G01N 33/50

(52) U.S. Cl. 702/20

(57) ABSTRACT

The invention relates to a method for registering, identifying and processing of data related to chemical compounds including the steps of: (a) entering information on structure and properties of known chemical compounds into a first group of data bases; (b) entering information on genes, including alleles, RNAs and proteins into a second group of data bases; (c) entering information on known diseases and on epidemiology into a third group of data bases; (d) entering information on data relating to individual living organisms into a fourth group of data bases; (e) entering information on any known links between any of the data contained in any of the first, second, third and forth groups of data bases into a fifth group of data bases; (f) developing a tool for mining and visualizing of data; and (g) applying the tool for mining and visualizing of data to generate new knowledge on effects of chemical compounds, alleles and gene products on given individual living organisms.

### BACKGROUND OF THE INVENTION

1. Field of the Invention

This application is a continuation-in-part application of U.S. application Ser. No. 10/005,666 filed on Nov. 2, 2001. The invention relates to a method for registering, identifying and processing data related to chemical compounds including drug specific data and for making drugs available to individual patients avoiding adverse drug reactions (ADR).

2. Description of the Prior Art

Current methods and systems being used for access control of drug delivery are not sufficiently reliable and secure for the routine medical treatment of patients with drugs.

Thus, more than 2 million adverse drug reactions occur annually in the United States. An analysis of 39 prospective studies (JAMA 1998, 279, 1200-1205) shows that in 1994 2,216,000 patients have been hospitalized with adverse drug reactions, 106,000 patients of them with fatal outcome.

Recent examples of drugs affected by severe ADR include the following:

| Drug | | Reaction |
|---|---|---|
| Troglitazone | Rezulin | Anti-diabetes type II drug; severe liver |
| | | toxicity, unexpected deaths; Warning |
| | | labeling introduced by FDA; withdrawn from |
| | | market |
| Trovafloxacin | Trovan | Antibiotic drug; unexpected severe liver |
| | | toxicity with deaths occurring; call for ban |
| | | of product that made US$ 68 Mio in its first |
| | | year in the USA alone; boxed warning |
| | | introduced by FDA. |
| Tolcapone | Tasmar | Anti-Parkinson drug; severe liver |
| | | toxicity with deaths; banned in UK, severely |
| | | restricted in its use the in us. |
| Lazabemide | Tempium | Anti-Alzheimer disease drug; severe liver |
| | | toxicity in Phase III clinical trials; |
| | | development aborted. |
| Sparfloxacin | Zagam | Antibiotic drug; severe phototoxicity |
| | | and cardiotoxicity; in EU limited to use in |
| | | pneumonia. |
| Grepafloxacin | Raxar | Antibiotic drug; severe cardiovascular |
| | | events, several deaths; withdrawn from |
| | | market. |
| Moxifloxacin | Avelox | Antibiotic drug; FDA panel split about |
| | | concerns on product safety; potential for |
| | | prolonged QT-interval; approved. |

### SUMMARY OF THE INVENTION

The challenge of the invention is to develop a new system usable for a controlled drug development and personalized drug delivery.

It is the object of the invention to develop a new sophisticated postgenomic knowledge management system which links chemical molecular modeling, bioinformatic, genetic, epidemiology, and molecular diagnostic data in order to develop prospective theragenomic information which allows the safe use of a given drug or a safe drug therapy, which is free of severe adverse drug reactions in each individual patient.

This object is achieved by a system for registering, identifying and processing of drug specific data, which links, among others, chemical, molecular modeling, bioinformatic, genetic, epidemiological, and molecular diagnostic data to develop prospective theragenomic information. The system according to the invention comprises

a master database correlating patterns of gene expression and genetic polymorphisms with drug-induced i.e. drug-related adverse effects and drug structure,

a data-tool for structural and genetic fingerprints predictive for adverse effects in individual patients, and

means for coupling the master database and the predictive tool in such a way that electronically prospective theragenomic information can be developed which allows the safe use of a given drug or a safe drug therapy, which is, with high probability, free of adverse drug reactions in an individual patient, wherein the master database is in a form such that data records of the following type can be entered:

Basic drug information such as for example information to intermediates, metabolites, adducts, targets, mimics, pathways, 2D-structures, 3D-structures and similarities.

Clinical endpoint information such as for example drugs, type of endpoint, frequency.

Drug-induced effects by genes on, as for example, receptors, promoters, transcription factors, responsive elements, expression patterns, gene function, 3D-structure, adduct targets, and autoantigens.

Drug-induced effects to allelic variants, such as for example, SNP's, splice variants, and amplifications on function(s), 3D-structure, frequencies, ethnic differences, predictive power, selectivity and sensitivity.

The object is also achieved by a method coupled with said system, wherein the master database is being coupled to the database of the predictive data-tool in such a way that a user of the system can develop and carry out different screening approaches either to verify the sociability of drugs for a specific selected category of patients or to search a specific drug for a selected category of patients which do not have adverse drug reactions or to make risk-analyses.

It is also the challenge of the invention to develop a method for registering, identifying and processing data related to chemical compounds including drug specific data and for making drugs available to individual patients avoiding adverse drug reactions (ADR). In such a method multiple groups of databases (e.g. five groups of databases) are interlinked.

This object is achieved by a method for registering, identifying and processing data related to chemical compounds comprising the steps of:

(a) entering information on structure and properties of known chemical compounds into a first group of data bases;

(b) entering information on genes, including alleles, RNAs and proteins into a second group of data bases;

(c) entering information on known diseases and on epidemiology into a third group of data bases;

(d) entering information on data relating to individual living organisms into a fourth group of data bases;

(e) entering information on any known links between any of the data contained in any of the first, second, third and forth groups of data bases into a fifth group of data bases;

(f) developing a tool for mining and visualizing of data; and

(g) applying the tool for mining and visualizing of data to generate new knowledge on effects of chemical compounds, alleles and gene products on given individual living organisms.

### DESCRIPTION OF THE INVENTION

It is generally known that the development of most drug-related adverse effects in man is based on the genetic and epigenetic predisposition, i.e. susceptibility of each individual. This predisposition is reflected in predictive patterns of structural properties, genetic polymorphisms and gene expression profiles that correlate with the development of drug related adverse effects.

The genetic predisposition is phenotypically revealed only when the individual carrier is exposed to (the) offending agent(s) or structural mimics thereof.

According to the present invention, the master database is stored on a separate server. In accordance with the invention the system also comprises a predictive data-tool in electronic form. In this tool the structural and genetic fingerprints predictive for given adverse effects in individual patients due to treatment with a selected drug are stored.

Each individual patient-specific data in reality comprises a set of selected yet predictive structural and genetic information that can be presented on a gene chip to be used in theratyping of individual patients.

This means that a given drug or a given combination of drugs used as therapy regimen have its corresponding unique set of patient-specific data. These data can be further classified into various subgroups as for example in subgroups dependent of the sex and/or of the age of the patients or in subgroups corresponding to clinical endpoints and/or risk groups.

A method according the invention is characterized in that the master database is being coupled to the database of the predictive data-tool in such a way that a user of the system can develop different screening approaches either to verify the sociability of drugs for a specific selected category of patients or to search a specific drug for a selected category of patients which do not have adverse drug reactions or to make risk-analyses.

Therefore, it is also part of the invention that with an adequate tool available, theragenomics, can entirely be done electronically.

It is another part of the invention to make available the system, i.e. the basic database and the predictive-tool data-base to the user via the Internet or an Intranet. For that purpose the system further comprises means which allow the user to follow up their screening procedures in the databases via an Internet- or Intranet-server, from where they can be called up merely by means of a login and a password.

The database type on which the method and system according to the invention is based is freely selectable and, for example, VISUAL FOXPRO. The same also applies to the computer operating system on which the database is based, which may be, for example, WINDOWS NT.

In another part of the invention multiple groups of databases are used. This is achieved by a method for registering, identifying and processing data related to chemical compounds comprising the steps of:

(a) entering information on structure and properties of known chemical compounds into a first group of data bases;

(b) entering information on genes, including alleles, RNAs and proteins into a second group of data bases;

(c) entering information on known diseases and on epidemiology into a third group of data bases;

(d) entering information on data relating to individual living organisms into a fourth group of data bases;

(e) entering information on any known links between any of the data contained in any of the first, second, third and forth groups of data bases into a fifth group of data bases;

(f) developing a tool for mining and visualizing of data; and

(g) applying the tool for mining and visualizing of data to generate new knowledge on effects of chemical compounds, alleles and gene products on given individual living organisms.

### BRIEF DESCRIPTION OF THE DRAWINGS

The invention will become apparent from the study of the following specification with reference to the attached drawing. In the drawing,

FIG. 1 illustrates the principle of the correlation of drug-dependent data stored in the master database and in the predictive data-tool, and

FIG. 2 shows a diagram illustrating the principle of the function of the system and method according to the invention.

### DETAILED DESCRIPTION OF THE DRAWINGS

The main features of the invention are:

The master database: Which is

an object-oriented and relational database-system,

based on a knowledge management-system,

Internet- or Intranet-based.

First group of databases Which comprises:

information structure and properties of known chemical compounds (e.g., drugs)

the properties include physical, chemical, pharmacokinetic, pharmacodynamic and toxicological properties.

Second group of databases Which comprises:

information on genes, including alleles, RNAs and proteins

the information includes pharmacogenetic, toxicogenetic, expression genetic and epigenetic data, splicing, DNA, RNA and protein modification

Third group of databases Which comprises:

information on known diseases and on epidemiology

Fourth group of databases Which comprises:

information on data relating to individual living organisms (e.g., human beings)

Fifth group of databases Which comprises:

information on any known links between any of the data contained in any of the first, second, third and forth groups of data bases

The predictive data-tool(s) Which comprises:

structural and genetic fingerprints (information), transferable to

diagnostic tools (for example, gene chip arrays).

The custom services: Which is characterized by consulting for example

pharmaceutical industries

regulatory agencies (such as for example BAG, IKS, BPharm, EMEA, FDA),

societies (such as for example SOT, EUROTOX, others),

patients, physicians, health care providers,

financial analysts, investors, lawyers, courts and

The public.

Tool for mining and visualizing date the mining of data includes editing, importing, annotating, arranging and visualization of information as well as path-finding, neighbor-finding, similarity searches and machine learning

Tool for generating new Which comprises: knowledge on effects of prediction of adverse drug reactions; chemical compounds, prediction of at least one specific drug suitable for alleles and gene products therapy in a given individual living organism; on given individual living organisms by applying the-prediction of at least one specific chemical compound tool for mining and to which exposure of a given individual living visualizing organism should be avoided;

prediction of possible new drug candidates suitable for treating one of a given disease and condition; and

identification of one of molecules and molecular domains occurring in a given individual living organism as targets for drugs

The master database correlates patterns of gene expression and genetic polymorphisms with drug-induced adverse effects and drug structure. This database can comprise drug specific date as follows:

Structural Chemistry and Genomics: Factors such as for example 2D-and 3D-structures; effects to receptors; ligands; metabolites; intermediates; function and structure of wt-gene and allelic variants gene products; chemical and postranslational modifications; others.

Pharmacogenetics: Factors such as for example mutated or re-arranged genes: e.g., CYP's; NAT-1; NAT-2; GST--; p53; Rb: WTI; BRCA1; BRCA2; VHL; APC; NF-1; NF-2; MYS-1; splice variants (i.e., CD44v5, CD44v6); transcription factors; responsive elements; many others.

Expression genetics: Factors such as for example over- or under-expression of unmutated or mutated genes: e.g., CYP's; p15; p107; p300; cyclin D1 (amplification); class II cancer genes (altered in expression); regulated transcription factors; many others.

Proteomics Factors such as for example up- or down-regulation of protein expression; posttranslational modifications; chemical adduct formation; gain of new properties through modification; others.

Epigenetic networks and Environmental factors: Other factors such as for example DNA-methylation; signalling cascades; narangenin; bergotamine, alkaloids; retinoids; quinines; co-medications; man-made chemicals in the environment; infections; viral loads; status or type of disease.

According to the invention, the input of data into the master database is effected by data-transfer from clinical centers, i.e from clinical data networks and/or from public domain, Internet and proprietor databases and/or from pharmaceutical companies and/or from bioinformatic databases. The data-transfer can ensure upon copying the data from diskettes, CD-ROM or DVD. Of course, it is also possible to transfer the data via the Internet.

The same procedure is effective for the set up of the predictive-data-tool.

The method according the invention couples the master database and the database of the predictive data-tool in such a way that a user of the system can develop different screening approaches either to verify the sociability of drugs for a specific selected category of patients or to search a specific drug, which show no adverse drug reactions for a selected category of patients, or to make risk-analyses.

The invention provides a method for registering, identifying and processing data related to chemical compounds comprising the steps of:

(a) entering information on structure and properties of known chemical compounds into a first group of data bases;

(b) entering information on genes, including alleles, RNAs and proteins into a second group of data bases;

(c) entering information on known diseases and on epidemiology into a third group of data bases;

(d) entering information on data relating to individual living organisms into a fourth group of data bases;

(e) entering information on any known links between any of the data contained in any of the first, second, third and forth groups of data bases into a fifth group of data bases;

(f) developing a tool for mining and visualizing of data; and

(g) applying the tool for mining and visualizing of data to generate new knowledge on effects of chemical compounds, alleles and gene products on given individual living organisms.

It should be noted that the chemical compounds, in steps (a) and (g) can include drugs. The properties, in step (a), can include physical, chemical, pharmacokinetic, pharmacodynamic and toxicological properties. The information, in step (b), can include pharmacogenetic, toxicogenetic, expression genetic and epigenetic data, splicing, DNA, RNA and protein modification. The individual living organisms referred to in steps (d) and (g) can include human beings.

The mining of data referred to in step (g), can include editing, importing, annotating, arranging and visualization of information as well as path-finding, neighbor-finding, similarity searches and machine learning. The knowledge on effects of chemical compounds on given individual living organisms referred to in step (g) can include

prediction of adverse drug reactions;

prediction of at least one specific drug suitable for therapy in a given individual living organism;

prediction of at least one specific chemical compound to which exposure of a given individual living organism should be avoided;

prediction of possible new drug candidates suitable for treating one of a given disease and condition; and

identification of one of molecules and molecular domains occurring in a given individual living organism as targets for drugs.

Screening approaches can follow up according to the table below:

| Clinical Endpoint(s), | Approaches, such as, for |
|---|---|
| such as, for example | example: |
| Hepatitis C: Response to | Pharmacogenetics |
| Therapy | Pattern of genetic polymorphism |
| Transplantation: Chronic | Pattern of gene expression |
| rejection | Biostatistics |
| Ulcerogenesis of NSAID's | Predictive Power |
| COX-1/2 | Sensitivity |
| Haemolytic Anaemia | Selectivity |
| Cholestasis | Pattern Frequency |
| Hepatitis | (Allele) Frequency |
| Thrombocytopenia | Level of confidence |
| Agranulocytosis | Molecular Modeling |
| | Chemical structure |
| | Structural similarity |
| | Molecular mimicry |
| | Homology modeling |

As mentioned above it is also a part of the invention to make available the system, i.e. the basic database and the predictive-tool database, to the public via the Internet. Preferred users are pharmaceutical industries, regulatory agencies, societies, patients, physicians and health care providers. Large pharmaceutical industries can use the system for postmarketing as well as for late and early drug-development purpose. Small pharmaceutical industries can use the system for compound selection and very early drug-development purpose.

The system and method according to the invention has enormous advantages. Thus, the pharmaceutical industries can screen their new products for adverse drug reactions in a simple and secure manner. Moreover, the data of the two data-bases of the system can be relatively simply updated and can be offered to various users on a data medium or by Internet or on Intranet. All in all, the method according to the invention also simplifies the search tasks of the industries to find out possible adverse reactions of drugs in development, so that it can, with high probability, be avoided that a drug has do be withdrawn from the market because of adverse drug reactions after coming onto the market.

What is claimed is:

1. A method for registering, identifying and processing data related to chemical compounds comprising the steps of:
(a) entering information on structure and properties of known chemical compounds into a first group of data bases;
(b) entering information on genes, including alleles, RNAs and proteins into a second group of data bases;
(c) entering information on known diseases and on epidemiology into a third group of data bases;
(d) entering information on data relating to individual living organisms into a fourth group of data bases;
(e) entering information on any known links between any of the data contained in any of the first, second, third and forth groups of data bases into a fifth group of data bases;
(f) developing a tool for mining and visualizing of data; and
(g) applying the tool for mining and visualizing of data to generate new knowledge on effects of chemical compounds, alleles and gene products on given individual living organisms.

2. The method of claim 1, wherein, in step (a), the chemical compounds include drugs.

3. The method of claim 1, wherein, in step (g), the chemical compounds include drugs.

4. The method of claim 1, wherein, in step (a), the properties include physical, chemical, pharmacokinetic, pharmacodynamic and toxicological properties.

5. The method of claim 1, wherein, in step (b), the information includes pharmacogenetic, toxicogenetic, expression genetic and epigenetic data, splicing, DNA, RNA and protein modification.

6. The method of claim 1, wherein, in step (d), the individual living organisms include human beings.

7. The method of claim 1, wherein, in step (g), the individual living organisms include human beings.

8. The method of claim 1, wherein, in step (g), wherein the mining of data includes editing, importing, annotating, arranging and visualization of information as well as path-finding, neighbor-finding, similarity searches and machine learning.

9. The method of claim 1, wherein, in step (g), the knowledge on effects of chemical compounds on given individual living organisms includes
prediction of adverse drug reactions;
prediction of at least one specific drug suitable for therapy in a given individual living organism;
prediction of at least one specific chemical compound to which exposure of a given individual living organism should be avoided;
prediction of possible new drug candidates suitable for treating one of a given disease and condition; and
identification of one of molecules and molecular domains occurring in a given individual living organism as targets for drugs.
